(19) United States
(12) Patent Application Publication
(10) Pub. No.: US 2026/0090914 A1
(43) Pub. Date: Apr. 2, 2026

(54) ADJUSTABLE SHUNTS WITH IMPROVED FLOW CONTROL AND ASSOCIATED SYSTEMS AND METHODS

(71) Applicant: Shifamed Holdings, LLC, Campbell, CA (US)

(72) Inventors: David Batten, Campbell, CA (US); Tessa Bronez, San Francisco, CA (US)

(21) Appl. No.: 19/120,217

(22) PCT Filed: Nov. 1, 2023
(86) PCT No.: PCT/US2023/078333

§ 371 Date: Apr. 10, 2025

Related U.S. Application Data
(60) Provisional application No. 63/421,851, filed on Nov. 2, 2022.

(51) Int. Cl. A61F 9/007

(52) U.S. Cl. CPC A61F 9/00781

(57) ABSTRACT

The present technology is generally directed to adjustable shunting systems, including adjustable shunting systems with improved flow control. For example, the adjustable shunting systems described herein can include an aperture formed within a flexible membrane extending over a well or other empty space. The flexible membrane can at least partially flex into the well or empty space to improve a seal at the aperture.

### CROSS-REFERENCE TO RELATED APPLICATION(S)

This application claims priority to U.S. Patent Application No. 63/421,851, filed Nov. 2, 2022, which is herein incorporated by reference in its entirety.

### TECHNICAL FIELD

The present technology generally relates to implantable medical devices and, in particular, to adjustable shunts for controlling fluid flow between a first body region and a second body region of a patient.

### BACKGROUND

Implantable shunting systems are widely used to treat a variety of patient conditions by shunting fluid from a first body region/cavity to a second body region/cavity. For example, shunting systems have been proposed for treating glaucoma. The flow of fluid through the shunting systems is primarily controlled by the pressure gradient across the shunt and the physical characteristics of the flow path defined through the shunt (e.g., the resistance of the shunt lumen). Conventional, early shunting systems (sometimes referred to as minimally invasive glaucoma shunts or “MIGS”) have shown clinical benefit; however, there is a need for improved shunting systems and techniques for addressing elevated intraocular pressure and risks associated with glaucoma, as well as other patient conditions. For example, there is a need for shunting systems capable of adjusting the therapy provided, including the flow rate/fluid resistance between the two fluidly-connected bodies. As another example, there is a need for a shunting system capable of being modified after manufacture (e.g., in the clinic) to personalize the system for the patient and/or as part of the clinician's plan for the implant procedure.

### BRIEF DESCRIPTION OF THE DRAWINGS

Many aspects of the present technology can be better understood with reference to the following drawings. The components in the drawings are not necessarily drawn to scale. Instead, emphasis is placed on illustrating clearly the principles of the present technology. Furthermore, components can be shown as transparent in certain views for clarity of illustration only and not to indicate that the component is necessarily transparent. Components may also be shown schematically.

FIG. 1A illustrates an adjustable shunting system configured in accordance with select embodiments of the present technology.

FIG. 1B is an exploded view of the adjustable shunting system of FIG. 1A.

FIG. 1C is an enlarged exploded view of an actuation assembly of the adjustable shunting system of FIGS. 1A and 1B.

FIG. 2A is an enlarged view of an actuator of the adjustable shunting system shown in FIGS. 1A and 1B and configured in accordance with select embodiments of the present technology.

FIGS. 2B and 2C illustrate embodiments of a sealing assembly of the actuator shown in FIG. 2A and configured in accordance with select embodiments of the present technology.

FIGS. 3A and 3B illustrate select features of the adjustable shunting system of FIGS. 1A and 1B in various fluid control states and configured in accordance with select embodiments of the present technology.

FIGS. 4A and 4B illustrate an additional embodiment of select features of the adjustable shunting system of FIGS. 1A and 1B in various fluid control states and configured in accordance with select embodiments of the present technology.

FIGS. 5A-5C illustrate another embodiment of select features of the adjustable shunting system of FIGS. 1A and 1B in various fluid control states and configured in accordance with embodiments of the present technology.

FIGS. 6A-6E schematically illustrate top views of portions of flexible membranes configured in accordance with additional embodiments of the present technology.

FIG. 7 is a schematic, cross-sectional side view of select features of an adjustable shunting system configured in accordance with embodiments of the present technology.

FIGS. 8A-8C schematically illustrate top views of portions of flexible membranes configured in accordance with further embodiments of the present technology.

FIG. 9 is a schematic, cross-sectional side view of select features of an adjustable shunting system configured in accordance with another embodiment of the present technology.

### DETAILED DESCRIPTION

The present technology is generally directed to adjustable shunting systems, including adjustable shunting systems with improved flow control. For example, the adjustable shunting systems described herein can include a shunting element with at least one channel extending therethrough that permits fluid to flow through the system. The system can further include an actuator that can be selectively actuated to control the flow of fluid through an inlet or outlet aperture of the channel to titrate the level of therapy provided by the shunt. In some embodiments, the actuator can include a gating element having a sealing assembly moveable between a first (e.g., open) position in which the sealing assembly provides a first resistance to fluid flow through the aperture (e.g., by not blocking the aperture), and a second (e.g., closed or partially closed) position in which the sealing assembly provides a second resistance greater than the first resistance (e.g., by at least partially blocking the aperture).

To further improve the flow control provided by the sealing assembly, the aperture can be formed within a flexible membrane that extends over a well, cavity, or other empty space. As the sealing assembly moves from the first (e.g., open) position toward the second (e.g., closed) position, the sealing assembly can cause the flexible membrane to flexibly bow or deform into the well. As described in detail through this Detailed Description, this is expected to improve the seal formed at the aperture when the sealing assembly is in the second (e.g., closed position) to provide for greater control of fluid flow through the adjustable shunting system.

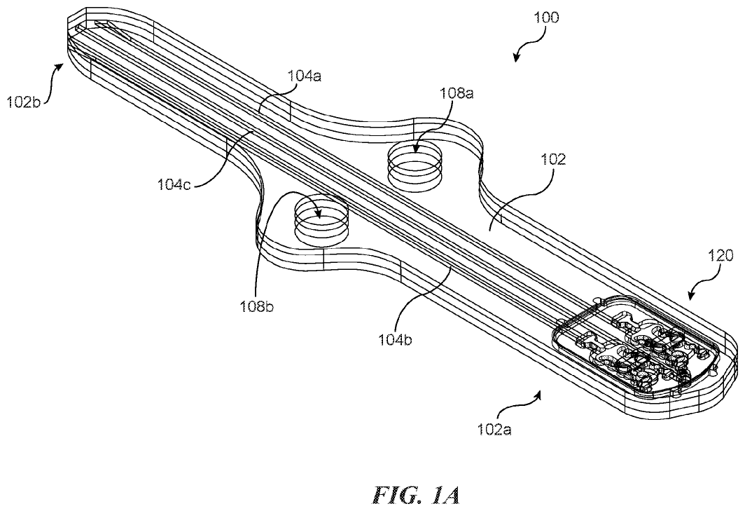

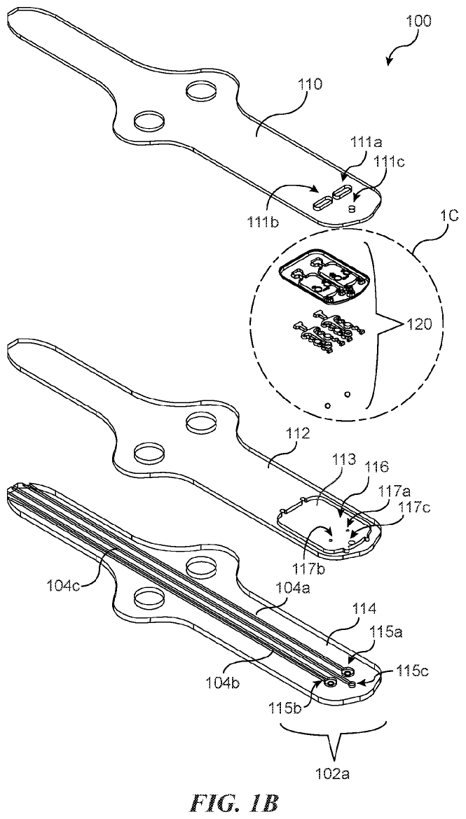

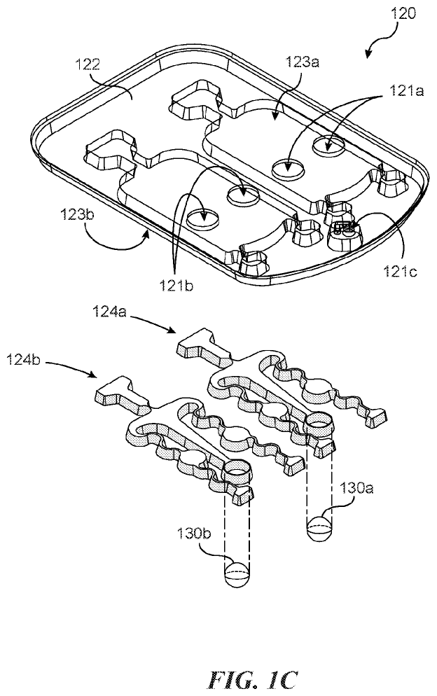

The terminology used in the description presented below is intended to be interpreted in its broadest reasonable manner, even though it is being used in conjunction with a detailed description of certain specific embodiments of the present technology. Certain terms may even be emphasized below; however, any terminology intended to be interpreted in any restricted manner will be overtly and specifically defined as such in this Detailed Description section. Additionally, the present technology can include other embodiments that are within the scope of the examples and claims but are not described in detail with respect to FIGS. 1A-9.

Reference throughout this specification to “one embodiment” or “an embodiment” means that a particular feature, structure, or characteristic described in connection with the embodiment is included in at least one embodiment of the present technology. Thus, the appearances of the phrases “in one embodiment” or “in an embodiment” in various places throughout this specification are not necessarily all referring to the same embodiment. Furthermore, the particular features or characteristics may be combined in any suitable manner in one or more embodiments.

As used herein, the use of relative terminology, such as “about”, “approximately”, “substantially” and the like refer to the stated value plus or minus ten percent. For example, the use of the term “about 100” refers to a range of from 90 to 110, inclusive. In instances in which the context requires otherwise and/or relative terminology is used in reference to something that does not include a numerical value, the terms are given their ordinary meaning to one skilled in the art.

Reference throughout this specification to the term “resistance” refers to fluid resistance unless the context clearly dictates otherwise. The terms “drainage rate” and “flow rate” are used interchangeably to describe the movement of fluid through a structure at a particular volumetric rate. The term “flow” is used herein to refer to the motion of fluid, in general.

Although certain embodiments herein are described in terms of shunting fluid from an anterior chamber of an eye, one of skill in the art will appreciate that the present technology can be readily adapted to shunt fluid from and/or between other portions of the eye or, more generally, from and/or between a first body region and a second, different body region of a patient. Moreover, while the certain embodiments herein are described in the context of glaucoma treatment, any of the embodiments herein, including those referred to as “glaucoma shunts” or “glaucoma devices” may nevertheless be used and/or modified to treat other diseases or conditions, including other diseases or conditions of the eye or other body regions. For example, the systems described herein can be used to treat diseases characterized by increased pressure and/or fluid build-up, including but not limited to heart failure (e.g., heart failure with preserved ejection fraction, heart failure with reduced ejection fraction, etc.), pulmonary failure, renal failure, hydrocephalus, and the like. Moreover, while generally described in terms of shunting aqueous, the systems described herein may be applied equally to shunting other fluid, such as blood or cerebrospinal fluid, between the first body region and the second body region.

FIGS. 1A-1C illustrate an adjustable shunting system 100 (“the system 100”) configured in accordance with select embodiments of the present technology. More specifically, FIG. 1A is a perspective view of the system 100, FIG. 1B is an exploded view of the system 100, and FIG. 1C is an enlarged view of an actuation assembly 120 of the system 100 as shown in FIG. 1B. As described in greater detail below, the system 100 is configured to provide a titratable therapy for shunting fluid from a first body region to a second body region, such as shunting aqueous from an anterior chamber of a patient's eye to a target outflow location.

Referring first to FIG. 1A, the system 100 includes a shunting element 102 and an actuation assembly 120. The shunting element 102 (which can also be referred to as a casing, membrane, elongated housing, or the like) extends between a first end portion 102a and a second end portion 102b. A plurality of flow channels 104 (shown as a first channel 104a, a second channel 104b, and a third channel 104c) can extend through the shunting element 102 at least partially between the first end portion 102a and the second end portion 102b. The channels 104 can be fluidly isolated along a portion or substantial portion of the length of the shunting element 102. As described in greater detail below, when the system 100 is implanted within a patient between a first body region and a second body region, fluid can flow from the first body region to the second body region via the channels 104. The shunting element 102 may optionally include one or more features to facilitate anchoring the system 100 to patient tissue, such as first and second suture holes 108a, 108b. The shunting element 102 can be composed of a partially flexible and/or biocompatible material, such as silicone, polydimethylsiloxane (PDMS), polymethylmethacrylate (PMA), or the like. For example, the shunting element 102 may be composed of a material having a durometer of between about 60 and about 90, or between about 70 and 80, or about 75. Additional features of shunting elements suitable for use with the present technology are described in International Patent Application No. PCT/US2022/037747, the disclosure of which is incorporated by reference herein in its entirety and for all purposes.

The actuation assembly 120 can be positioned within the first end portion 102a of the shunting element 102. As described in greater detail below, the actuation assembly 120 can have one or more features that control the flow of fluid through one or more of the channels 104. In this way, the actuation assembly 120 can be selectively manipulated by a user to adjust the resistance through the system 100, and thus the level of therapy provided by the system 100.

Referring next to FIG. 1B, the shunting element 102 can include a plurality of layers that are stacked and sealed together to collectively form the shunting element 102. For example, the shunting element 102 can include a first (e.g., top) layer 110, a second (e.g., middle) layer 112, and a third (e.g., bottom) layer 114. Accordingly, in the illustrated embodiment the shunting element 102 includes three layers, although in other embodiments the shunting element 102 can include more or fewer layers, such as one, two, four, five, or six layers. In operation, the first layer 110, the second layer 112, and the third layer 114 are sealed together (e.g., glued, adhered, bonded, etc.) to form the shunting element 102. More specifically, a lower surface of the first layer 110 is sealed to an upper surface of the second layer 112, and a lower surface of the second layer 112 is sealed to an upper surface of the third layer 114. Sealing the layers prevents or at least reduces fluid from leaking through the system 100 between layers. Additional details regarding multi-layered shunting systems are described in International Patent Application No. PCT/US2022/037917, the disclosure of which is incorporated by reference herein in its entirety.

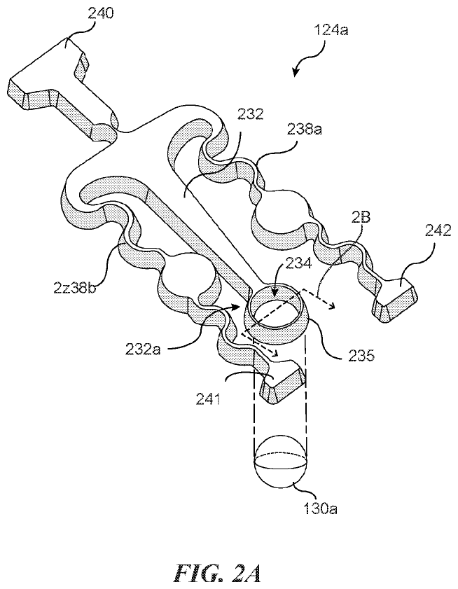

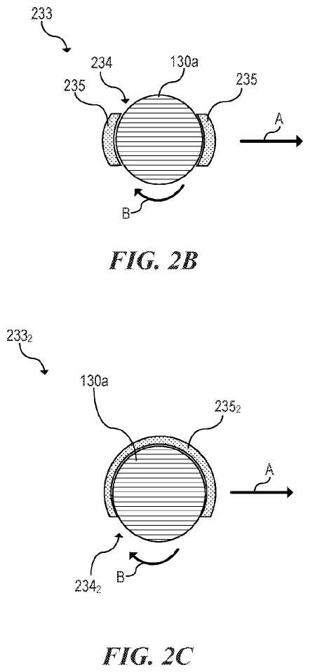

The first layer 110 includes several openings (e.g., windows, ports, apertures, etc.). More specifically, the first layer 110 includes a first opening 111a, a second opening 111b, and a third opening 111c (collectively referred to as the openings 111). The openings 111 can have the same or different shapes and/or sizes. For example, in the illustrated embodiment, the first opening 111a and the second opening 111b have a generally similar shape and size, while the third opening 111c has a different shape (e.g., round vs. oval) and size (e.g., smaller). In operation, the openings 111 permit fluid to flow into the system 100. More specifically, and as described in greater detail below, the first opening 111a permits fluid to flow into the first channel 104a, the second opening 111b permits fluid to flow into the second channel 104b, and the third opening 111c permits fluid to flow into the third channel 104c. In addition to permitting fluid to flow into the system, the openings 111 can enable a user to view and/or actuate the actuation assembly 120. For example, when the system 100 is in an assembled configuration, the first opening 111a can be at least partially aligned with a first actuator 124a of the actuation assembly 120, and the second opening 111b can be at least partially aligned with a second actuator 124b of the actuation assembly 120. As described in greater detail with reference to FIG. 2A, during operation of the system 100, a user can actuate the first actuator 124a or the second actuator 124b by directing energy (e.g., laser energy) through the first opening 111a or the second opening 111b, respectively.

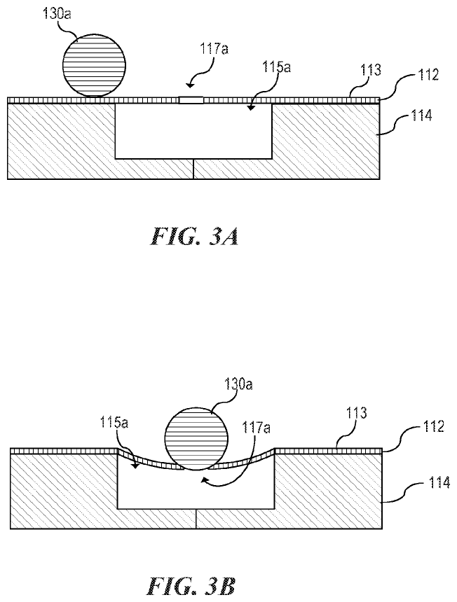

The second layer 112 includes a chamber or cavity 116 at the first end portion 102a, with an opening to the chamber 116 facing toward the first layer 110. The second layer 112 therefore has a flexible membrane 113 forming the lower surface of the chamber 116. The membrane 113 is defined, at least in part, by a thinned portion/region of the second layer, as compared to a thickness of the second layer 112 elsewhere along the length of the second layer 112. For example, the second layer 112 (outside of the thinned portion of the membrane 113) may have a thickness of between about 80 micron and about 200 micron, but the membrane 113 at the chamber 116 may have a reduced thickness of between about 20 micron and about 80 micron, or between about 30 micron and about 70 micron, or between about 40 micron and about 60 micron, or about 50 micron. The foregoing ranges are provided by way of example only—in other embodiments, the second layer 112 and/or the thinned portion defining the membrane 113 can have thicknesses outside the foregoing ranges. The chamber 116 provides an empty space or cavity for receiving the actuation assembly 120. Moreover, as described in greater detail with reference to FIGS. 3A and 3B, the membrane 113 of the second layer 112 also assists in improving the fluid flow control that can be achieved using the actuation assembly 120.

The chamber 116 includes several openings (e.g., ports, apertures, etc.) that generally align with the openings 111 of the first layer 110. For example, the membrane 113 of the second layer 112 includes a first aperture 117a, a second aperture 117b, and a third aperture 117c (collectively referred to as the apertures 117). The apertures 117 extend fully through the second layer 112 such that fluid can flow through the second layer 112 via the apertures 117. Similar to the openings 111, the apertures 117 can have the same or different shapes and/or sizes. In the illustrated embodiment, the first aperture 117a and the second aperture 117b have generally the same shape and size, while the third aperture 117c has generally the same shape (e.g., round) but a larger size (e.g., diameter). The first aperture 117a is fluidly connected to both the first opening 111a and the first channel 104a such that fluid flowing into the system 100 via the first opening 111a can flow into the first channel 104a via the first aperture 117a. Similarly, the second aperture 117b is fluidly connected to both the second opening 111b and the second channel 104b, such that fluid flowing into the system 100 via the second opening 111b can flow into the second channel 104b via the second aperture 117b. The third aperture 117c is fluidly connected to both the third opening 111c and the third channel 104c such that fluid flowing into the system 100 via the third opening 111c can flow into the third channel 104c via the third aperture 117c.

The third layer 114 defines or at least partially defines the channels 104. For example, the void space of the channels 104 can be formed within the third layer 114, although the second layer 112 can form a “top” of the channels 104 (e.g., the channels become closed off once the second layer 112 is sealed to the third layer 114). The third layer 114 also defines a first well 115a fluidly coupled to the first channel 104a at the first end portion 102a, a second well 115b fluidly coupled to the second channel 104b at the first end portion 102a, and a third well 115c fluidly coupled to the third channel 104c at the first end portion 102a. The first well 115a is aligned with, and therefore configured to receive fluid from, the first aperture 117a of the second layer 112. The second well 115b is aligned with, and therefore configured to receive fluid from, the second aperture 117b of the second layer 112. The third well 115c is aligned with, and therefore configured to receive fluid from, the third aperture 117c. In the illustrated embodiment, each of the wells 115 has a circular cross-sectional shape. In other embodiments, however, one or more of the wells 115 can have a different shape. For example, in some embodiments the first well 115a and/or the second well 115b has an oval shape and/or an elongated channel-like shape. In such embodiments, the elongated portion of the well 115 can extend generally normal to an axial length of the system 100, and may be at least partially curved.

Of note, at least the first well 115a and the second well 115b are bigger than the first aperture 117a and the second aperture 117b. For example, the first well 115a may have a first cross-sectional area that is greater than a second cross-sectional area of the first aperture 117a. In some embodiments, for example, the first cross-sectional area is between two to 50 times greater than, or between two to 25 times greater than, or between two to ten times greater than, the second cross-sectional area. Similarly, the first well 115a may have a first perimeter that is greater than a second perimeter of the first aperture 117a, such as between two to ten times greater than the second perimeter. The second well 115b may similarly have a greater cross-sectional area and/or perimeter than the second aperture 117b. As a result, some of the thinned portion 113 of the second layer 112 overlaps the first well 115a and the second well 115b. As described in greater detail below with reference to FIGS. 3A and 3B, this is expected to improve the seal that can be achieved by the first actuator 124a at the first aperture 117a and the second actuator 124b at the second aperture 117b.

As described above and as best shown in FIG. 1C, the actuation assembly 120 includes a first actuator 124a and a second actuator 124b (collectively referred to as the actuators 124). The first actuator 124a can be configured to selectively control the fluid resistance and/or flow of fluid through the first aperture 117a of the second layer 112 (and thus through the first channel 104a), and the second actuator 124b can be configured to selectively control the fluid resistance and/or the flow of fluid through the second aperture 117b of the second layer 112 (and thus through the second channel 104b). More specifically, the first actuator 124a can be selectively moveable between a first (e.g., open) position in which the first actuator 124a does not block or at least does not substantially block, and therefore permits fluid flow through, the first aperture 117a, and a second (e.g., closed or at least partially closed) position in which the first actuator 124a substantially blocks and/or seals, and therefore does not permit flow or at least clinically meaningful flow, through the first aperture 117a. That is, the first actuator 124a imparts a greater fluidic resistance through the first aperture 117a when the first actuator 124a is in the second position relative to when the first actuator 124b is in the first position. Likewise, the second actuator 124b can be selectively moveable between a first (e.g., open) position in which the second actuator 124b does not block or at least does not substantially block, and therefore permits fluid flow through, the second aperture 117b, and a second (e.g., closed or at least partially closed) position in which the second actuator 124b substantially blocks and/or seals, and therefore does not permit flow or at least clinically meaningful flow, through the second aperture 117b.

As also best shown in FIG. 1C, the actuation assembly 120 can also include a plate, cartridge, or backbone 122 configured to hold and prime the actuators 124 and positionable within the chamber 116 of the second layer 112. For example, the plate 122 can include a first actuator chamber 123a configured to receive the first actuator 124a and a second actuator chamber 123b configured to receive the second actuator 124b (collectively referred to as the actuator chambers 123; the openings to the actuator chambers 123 are facing downwardly toward, and thus configured to receive, the actuators 124 in the orientation shown in FIG. 1C). The actuator chambers 123 can be sized and shaped such that they at least partially deform (e.g., stretch, tension, compress, etc.) the actuators 124 when the actuators 124 are positioned therein. In embodiments in which the actuators 124 are composed of a shape memory material, this deformation primes the actuators 124 and permits them to be subsequently actuated, as described in greater detail with reference to FIG. 2A. The plate 122 also include one or more first plate openings 121a that generally align with the first opening 111a in the first layer 110, one or more second plate openings 121b that generally align with the second opening 111b in the first layer 110, and one or more third openings 121c that generally align with the third opening 111c in the first layer 110. As described below, the first plate openings 121a and the second plate openings 121b permit a user to actuate the corresponding actuators 124 (e.g., by providing a line-of-sight to a portion of the corresponding actuator 124), while each of the openings 121 permit fluid to flow through the system 100, as described below.

For example, in addition to housing the actuators 124, the actuator chambers 123 also form part of the fluid flow path through the system 100. For example, the first actuator chamber 123a is (a) fluidly coupled to the first opening 111a in the first layer 110 via the first plate opening(s) 121a, and (b) fluidly coupled to the first aperture 117a in the second layer 112, such that fluid can flow between the first opening 111a and the first aperture 117a via the first plate opening(s) 121a and the first actuator chamber 123a. Likewise, the second actuator chamber 123b is (a) fluidly coupled to the second opening 111b in the first layer 110 via the second plate opening(s) 121b, and (b) fluidly coupled to the second aperture 117b in the second layer 112, such that fluid can flow between the second opening 111b and the second aperture 117b via the second plate opening(s) 121b and second actuator chamber 123b. In some embodiments, the actuator chambers 123 are fluidly isolated. In other embodiments, the actuator chambers 123 are fluidly connected.

The plate 122 can be composed of a material that has generally stiffer mechanical properties than the layers 110, 112, 114, and/or the actuators 124. For example, the plate 122 can be composed of superelastic Nitinol, stainless steel, titanium, glass, plastic, or other suitable materials. This is expected to enable the plate 122 to resist deformation when the actuators 124 are deformed and coupled to the plate 122, as described in greater detail below. This feature is also expected to enable the plate 122 to resist upward deflection of the actuators 124, which can assist in improving fluid flow control through the system 100.

Each actuator 124 also includes a sealing element 130, which is shown separately from the actuators 124 in the exploded view for ease of illustration. More specifically, the first actuator 124a includes a first sealing element 130a and the second actuator 124b includes a second sealing element 130b. The sealing elements 130 can be composed of a generally noncompressible material such as glass, plastic, stainless steel, or the like. In other embodiments, the sealing elements 130 can be composed of a partially elastic material, such as silicone, rubber, or the like. As described in detail below with reference to FIGS. 3A and 3B, the sealing elements 130 can improve the fluid blocking effect (e.g., seal) of the actuators 124 at the corresponding first aperture 117a and second aperture 117b when the actuators 124 are in the closed position. Additional details of the sealing elements 130 are described below with reference to FIGS. 2A-3B.

FIG. 2A is an enlarged view of the first actuator 124a and the first sealing element 130a, with other aspects of the system 100 omitted for purposes of illustration. The first actuator 124a includes a projection or gating element 232 having a distal end portion 232a configured to at least partially control (e.g., gate) flow through the system 100. To do so, the distal end portion 232a includes a sealing assembly 233 configured to interface with a corresponding aperture 117 in the second layer 112 of the system 100 (FIG. 1B). The sealing assembly 233 includes a sealing element retention feature 235. In the illustrated embodiment, the sealing element retention feature 235 is an annular ring structure having an aperture 234 extending therethrough. In an assembled configuration, the first sealing element 130a is positioned within the aperture 234 and retained by the sealing element retention feature 235, as indicated in FIG. 2A.

FIG. 2B is a cross-sectional view of the sealing assembly 233 taken along the line labeled 2B in FIG. 2A and shows the first sealing element 130a retained within the sealing element retention feature 235. As shown in FIG. 2B, the aperture 234 can receive and retain the first sealing element 130a. In some embodiments, the first sealing element 130a is movable relative to (e.g., configured to rotate relative to) the sealing element retention feature 235. For example, as shown in FIG. 2B, the first sealing element 130a can have a spherical shape and be moveably and/or rotatably contained at least partially within the aperture 234 of the sealing element retention feature 235. In embodiments in which the first sealing element 130a is rotatable within the sealing element retention feature 235, as the sealing assembly 233 is translated in a first direction indicated by arrow A (e.g., via actuation of the first actuator 124a shown in FIG. 2A), the first sealing element 130a rotates in a second direction indicated by arrow B. Enabling rotation of the first sealing element 130a relative to the sealing element retention feature 235 may improve performance of the first actuator 124a because it permits the first sealing element 130a to rotate as it slidably moves along the thinned portion 113 of the second layer 112, reducing the frictional interference between the first sealing element 130a and the thinned portion 113. However, as described below, in some embodiments the first sealing element 130a is not rotatably moveable relative to sealing element retention feature 235.

The sealing assembly 233 can have a variety of suitable configurations. FIG. 2C, for example, illustrates another representative embodiment of a sealing assembly 233₂ configured in accordance with select embodiments of the present technology. Relative to the sealing assembly 233 described with reference to FIG. 2B, the sealing assembly 233₂ has a sealing element retention feature 235₂ that more fully encloses the first sealing element 130a. More specifically, the sealing element retention feature 235₂ defines a chamber 234₂ instead of an aperture. The first sealing element 130a can be moveably and/or rotatably retained within the chamber 234₂. Similar to the embodiment described with reference to FIG. 2B, the first sealing element 130a is configured to rotate in a first direction indicated by arrow B as the sealing assembly 233₂ is translated in a second direction indicated by arrow A.

Although FIGS. 2B and 2C described the first sealing element 130a as being moveably and/or rotatably coupled to the sealing element retention feature 235, in some embodiments the first sealing element 130a is non-moveably and/or non-rotatably coupled to the sealing element retention feature 235. In such embodiments, the first sealing element 130a can be coupled to the sealing element retention feature 235 via mechanical tethers, bands, barbs, staples, sutures, glue, chemical bonding, or the like, and/or the first sealing element 130a can be integrally formed with the distal end portion 232a of the gating element 232 such that the sealing element retention feature 235 is omitted. Moreover, although shown as having a spherical shape, in other embodiments the first sealing element 130a can have other suitable shapes, such as a conical shape, and oval-shape, etc.

Returning to FIG. 2A, the actuator 200 further includes a first actuation element 238a and a second actuation element 238b. The first actuation element 238a can be configured to rotate, pivot, slide, or otherwise move the gating element 232, and thus the sealing assembly 233, in a first direction. The second actuation element 238b can be configured to selectively rotate, pivot, slide, or otherwise move the gating element 232, and thus the sealing assembly 233, in a second direction generally opposite the first direction. For example, the first actuation element 238a and the second actuation element 238b can be composed at least partially of a shape memory material or alloy (e.g., Nitinol). Accordingly, the first actuation element 238a and the second actuation element 238b can be transitionable at least between a first material phase or state (e.g., a martensitic state, a R-phase, a composite state between martensitic and R-phase, etc.) and a second material phase or state (e.g., an austenitic state, an R-phase state, a composite state between austenitic and R-phase, etc.). In the first material state, the first actuation element 238a and the second actuation element 238b may have reduced (e.g., relatively less stiff) mechanical properties that cause the actuation elements to be more easily deformable (e.g., compressible, expandable, etc.) relative to when the actuation elements are in the first material state. In the second material state, the first actuation element 238a and the second actuation element 238b may have increased (e.g., relatively more stiff) mechanical properties relative to the first material state, causing an increased preference toward a specific preferred geometry (e.g., original geometry, manufactured or fabricated geometry, heat set geometry, etc.).

The first actuation element 238a and the second actuation element 238b can be selectively and independently transitioned between the first material state and the second material state by applying energy (e.g., laser energy, electrical energy, etc. delivered from an energy source external to the system 100 and a patient in which the system 100 is implanted) to the first actuation element 238a or the second actuation element 238b to heat the corresponding actuation element above a transition temperature (e.g., above an austenite finish (Af) temperature, which is generally greater than body temperature). If the first actuation element 238a (or the second actuation element 238b) is deformed relative to its preferred geometry when heated above the transition temperature, the first actuation element 238a (or the second actuation element 238b) will move to and/or toward its preferred geometry. In some embodiments, the first actuation element 238a and the second actuation element 238b are operably coupled such that, when the actuated actuation element (e.g., the first actuation element 238a) transitions toward its preferred geometry, the non-actuated actuation element (e.g., the second actuation element 238b) is further deformed relative to its preferred geometry. Additional details regarding, and examples of, bi-directional shape memory actuators that can be used with the present technology are described in U.S. Patent Application Publication Nos. 2020/0229982 and 2021/0251806 and U.S. Provisional Patent Application No. 63/392,695 , the disclosures of which are incorporated by reference herein in their entireties and for all purposes.

The first actuator 124a further includes a first anchoring element 240, a second anchoring element 241, and a third anchoring element 242 (collectively referred to as the anchoring elements 240-242). To couple the first actuator 124a to the system 100, the anchoring elements 240-242 can be secured to (e.g., placed within) corresponding anchoring features in the first actuator chamber 123a of the actuation assembly 120 (FIG. 2A). In some embodiments, the first actuation element 238a and the second actuation element 238b are deformed relative to their preferred geometries (e.g., “loaded”) when the first actuator 124a is positioned within the first actuator chamber 123a. For example, the first actuator chamber 123a can be configured/dimensioned such that the act of placing the anchoring elements 240-242 within the corresponding anchoring features deforms the first actuation element 238a and the second actuation element 238b relative to their original manufactured geometries. In some embodiments positioning the anchoring elements 240-242 within corresponding anchoring features can increase a length of the actuation elements 238 (e.g., tension) relative to their preferred geometries. In other embodiments, positioning the anchoring elements 240-242 within corresponding anchoring features can decrease a length of the actuation elements 238 (e.g., compress) relative to their preferred geometries. Additional details regarding loading and deforming shape memory actuators are described in U.S. Patent Application Publication No. US 2021/0251806, previously incorporated by reference herein, and International Patent Application No. PCT/US21/49140, the disclosure of which is incorporated by reference in its entirety and for all purposes.

As described above, the distal end portion 232a of the gating element 232 is configured to moveably interface with various features of the system 100 to at least partially control the flow of fluid through one or more flow pathways extending through the system 100. For example, referring collectively to FIGS. 1B and 2A, when the first actuator 124a is positioned within the first actuator chamber 123a, the distal end portion 232a of the gating element 232 is positioned proximate the first aperture 117a in the second layer 112 of the shunting element 102. As a result, the first actuator 124a can selectively move the sealing assembly 233 between the first (e.g., open) position in which the sealing assembly 233 does not block or substantially block flow through the first aperture 117a, and the second (e.g., closed) position in which the sealing assembly 233 blocks, or at least partially blocks, fluid flow through the first aperture 117a. In this way, the first actuator 124a can control the flow of fluid through the first channel 104a.

Flow through the second channel 104b can be controlled in the same or generally similar manner as flow through the first channel 104a. For example, the second actuator 124b can be the same as or generally similar to the first actuator 124a, but can be positioned within the second actuator chamber 123b such that the second actuator 124b is proximate the second aperture 117b in the second layer 112 of the shunting element 102. In contrast to the first channel 104a and the second channel 104b, the third channel 104c is designed to be “always open” such that it permits at least some degree of fluid flow through the system 100 even when both the first channel 104a and the second channel 104b are blocked/closed. Of course, the present technology is not limited to particular combinations of “always open” and adjustable channels, and can include more or fewer of each channel type. Similarly, although described as having two actuators 124, the system 100 can have more or fewer actuators, such as one, three, four, or more.

Without intending to be bound by theory, the sealing assembly 233, the membrane 113 (thinned portion) of the second layer 112, and the wells 115 in the third layer 114 are expected to improve the “seal” that can achieved at the first aperture 117a and the second aperture 117b when the first actuator 124a and the second actuator 124b, respectively, are in their second (e.g., closed) positions. For example, FIG. 3A is a schematic, cross-sectional illustration of the first sealing element 130a, the second layer 112, the third layer 114, and the plate 122 showing the first sealing element 130a in the first (e.g., open) position, and FIG. 3B is a schematic, cross-sectional illustration of the first sealing element 130a, the second layer 112, the third layer 114, and the plate 122 showing the first sealing element 130a in the second (e.g., closed) position. As one skilled in the art will appreciate, the schematic illustration of FIGS. 3A and 3B are intended to more clearly illustrate certain principles of the present technology, and thus are not necessarily drawn to scale and omit certain features of the system 100 for ease of illustration.

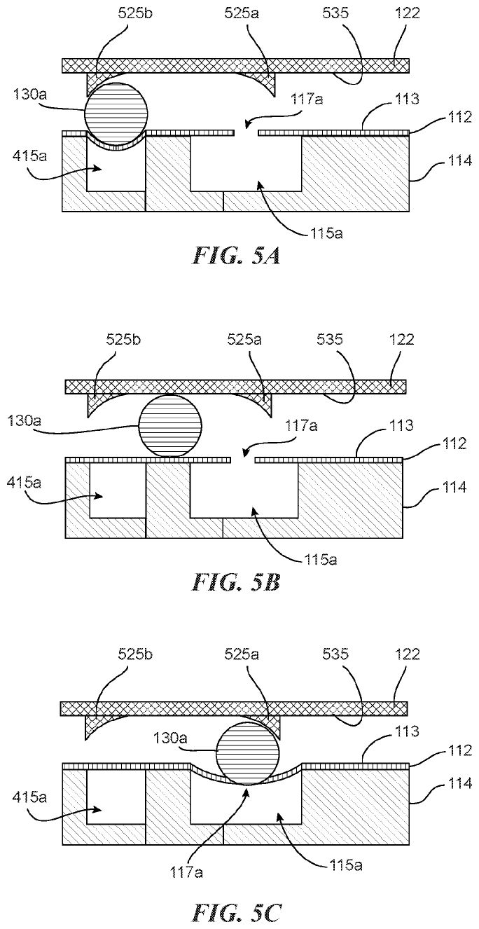

Referring first to FIG. 3A, when the first actuator 124a (FIGS. 1C and 2A) is in the first (e.g., open) position, the first sealing element 130a is positioned away from the first aperture 117a such that the first sealing element 130a does not block fluid from flowing through the first aperture 117a. Of note, when in the first (e.g., open) position, the first sealing element 130a does not overlap with the first well 115a (e.g., the first sealing element 130a is not positioned above the first well 115a). In contrast, as the first sealing element 130a moves toward the second (e.g., closed) position shown in FIG. 3B in response to actuation of the first actuator 124a, the first sealing element 130a moves over a region of the membrane 113 that does overlap with the first well 115a. The combination of the weight of the first sealing element 130a and the elasticity/flexibility of the thinned portion of the second layer 112 (the membrane 113) causes the membrane 113 to bow or flex downwardly into the empty space defined by the first well 115a. This downward bowing is expected to be advantageous because it helps direct the first sealing element 130a toward the first aperture 117a, which can be positioned at a center point or approximate center point above the first well 115a. This is also expected to be advantageous because it helps improve and maintain the seal at the first aperture 117a once the first sealing element 130a is in the second (e.g., closed) position. That is, because (a) the membrane 113 over the first well 115a becomes slightly concave in response to the first sealing element 130a moving over it, and (b) the first aperture 117a is positioned at the center (e.g., lowest point) of the concave region, the first sealing element 130a preferentially sits over the first aperture 117a. Without intending to be bound by theory, this is expected to improve the seal obtained at the first aperture 117a, which in turn is expected to improve the precision with which flow through the system 100 can be controlled. In some embodiments, such as described below with reference to FIGS. 5A-5C, the plate 120 may have one or more features that assist with directing the sealing element downward to flex the membrane 113 into the first well 115a.

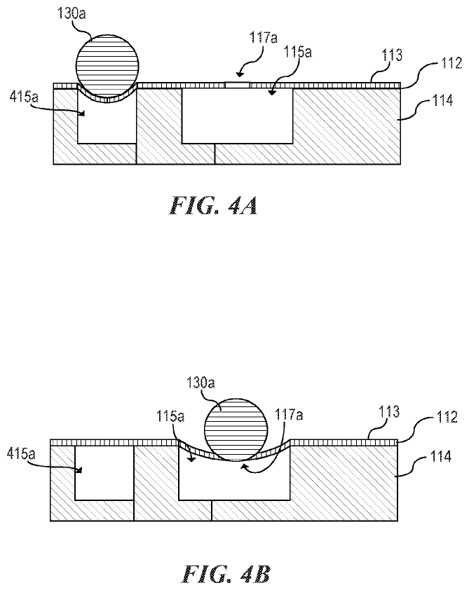

FIGS. 4A and 4B illustrate a variation of the system 100 that is expected to provide additional control over the position of the first sealing element 130a. Similar to FIGS. 3A and 3B, FIGS. 4A and 4B are schematic, cross-sectional illustrations of the first sealing element 130a, the second layer 112, the third layer 114, and the plate 120 in the first (e.g., open) position and the second (e.g., closed position). Relative to the embodiments shown and described with reference to FIGS. 3A and 3B, the embodiment shown in FIGS. 4A and 4B includes a second well or cavity 415a in the third layer 114. As best shown in FIG. 4A, when the first sealing element 130a is in the first (e.g., open) position, the first sealing element 130a is positioned generally above the second well 415a. Because the second cavity 415a is an empty space, and because the membrane 113 (the thinned portion of the second layer 112) is elastic, the weight of the first sealing element 130a causes the membrane 113 to bow downwardly into the second cavity 415a. This helps retain the first sealing element 130a in the first (e.g., open) position.

The operation of moving the first sealing element 130a from the first position (FIG. 4A) toward the second position (FIG. 4B) is generally the same as described with respect to FIGS. 3A and 3B. However, moving the first sealing element 130a between the first position and the second position requires passing through a higher energy state (e.g., as the first sealing element 130a jumps between the second well 415a and the first well 115a). In other words, the first sealing element 130a exists in a relatively stable, low energy state in both the first position and the second positions, and must pass through a less stable, higher energy state to transition therebetween. As a result, the first sealing element 130a is biased toward the first position and the second position, and will only move therebetween in response to an energy input (e.g., actuation of the first actuator 124a; FIG. 2A). Accordingly, the embodiment shown in FIGS. 4A and 4B is expected to enable a stable and consistent motion of the first sealing element 130a between the first (e.g., open) position and the second (e.g., closed) position during operation of the system.

FIGS. 5A-5C illustrate another variation of the system 100 that is expected to also provide additional control over the position of the first sealing element 130a. Similar to FIGS. 3A-4B, FIGS. 5A-5C are schematic, cross-section illustrations of the first sealing element 130a, the second layer 112, the third layer 114, and the plate 122. FIG. 5A illustrates the first sealing element 130a in the first (e.g., open) position, FIG. 5B illustrates the first sealing element 130a in an intermediate position between the first position and the second (e.g., closed) position, and FIG. 5C illustrates the first sealing element 130a in the second position. Relative to the embodiments shown and described with reference to FIGS. 3A-4B, plate 122 in the embodiment shown in FIGS. 5A and 5B includes a plurality of protrusions or ramping elements that assist with controlling the path of motion of the first sealing element 130a. More specifically, a lower surface 523 of the plate 122 includes a first protrusion or ramping element 525a configured to align (e.g., vertically align) with the first well 115a and a second protrusion or ramping element 525b configured to align (e.g., vertically align) with the second cavity 415b. As the first sealing element 130a moves from the first position (FIG. 5A) toward the second position (FIG. 5C), the first sealing element 130a engages with the first ramping element 525a. More specifically, as the first sealing element 130a is translated laterally toward the first well 115a, the first sealing element 130a rides along the first ramping element 525a. Because the first ramping element 525a is sloped toward the first well 115a, the first sealing element 130a is pushed into the membrane 113, which causes the membrane 113 to flex into the first well 115a, as previously described. That is, the first ramping element 525a provides an additional mechanism (e.g., in addition to gravity) that causes the first sealing element 130a to “fall into” the first well 115a. In this way, the first ramping element 525a is expected to improve the consistency of the seal between the first sealing element 130a and the first aperture 117a when the first sealing element 130a is in the second (e.g., closed) position. The second ramping element 525b can operate in a similar manner with respect to directing the first sealing element 130a toward, and retaining the first sealing element 130a at, the first position. For example, as the first sealing element 130a moves from the second position (FIG. 5C) toward the first (FIG. 5A), the first sealing element 130a engages with and rides along the second ramping element 525a, which pushes the first sealing element 130a at least partially into the second well 415a.

Although described in the context of having two ramping elements 525, in some embodiments the systems described herein have a single ramping element (e.g., a single ramping element aligned with the first well 115a, such as in the embodiment shown in FIGS. 3A and 3B). Similarly, the ramping elements 525 can have other structures beyond those illustrated. For example, in some embodiments the ramping elements 525 can be a sloped but generally uncurved surface. In other embodiments, the ramping elements 525 can include two-dimensional slots, such as those described in International Patent Application No. PCT/US23/20973, filed May 4, 2023, the disclosure of which is incorporated by reference herein in its entirety.

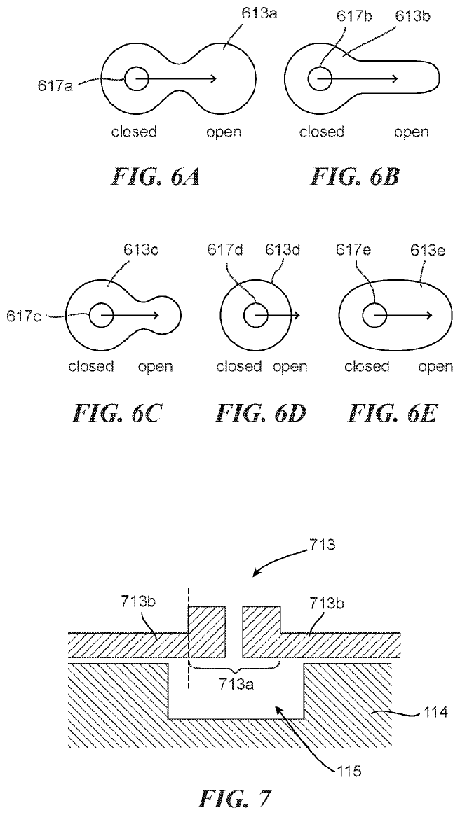

FIGS. 6A-6E schematically illustrate top views of portions of flexible membranes configured in accordance with additional embodiments of the present technology. The disclosed flexible membranes can be similar to those described above with reference to FIGS. 1B and 3A-5C. As with the membranes described previously, the flexible membranes of FIGS. 6A-6E comprises regions of the second layer 112 sized and shaped to extend over wells (e.g., first well 115a and well 415a) and include one or more apertures extending therethrough and configured to permit fluid to flow into the corresponding well. Without being bound by theory, it will be appreciated that the shape of the membranes affects both motion and sealing properties of the sealing element (e.g., sealing element 130) carried thereon.

FIG. 6A, for example, schematically illustrates a top view of a membrane 613a having an aperture 617a extending therethrough. In this embodiment, the membrane 613a comprises an hourglass shape. FIG. 6B illustrates a membrane 613b having an aperture 617b. The membrane 613b includes a first portion having a relatively circular cross-sectional shape and a second portion having a different shape (an elongated linear shape). FIG. 6C illustrates a membrane 613c having an aperture 617c at a first side of the membrane 613c. The membrane 613c includes a first portion having a large circular cross-sectional shape and a second portion having a much smaller circular cross-sectional shape. FIG. 6D illustrates a membrane 613d having an aperture 617d. The membrane 613d only includes a single circular portion/region. FIG. 6E illustrates a membrane 613e having an enlarged oval shape and including an aperture 617e. It will be appreciated that membranes configured in accordance with further embodiments of the present technology can have a variety of other suitable shapes/arrangements in addition to those shown in FIGS. 6A-6E.

FIG. 7 is a schematic, cross-sectional side view of a portion of a flexible membrane 713 configured in accordance with embodiments of the present technology. The membrane 713 is carried by the third layer 114 and positioned over well 115. The membrane 713 differs from those described previously in that membrane 713 has portions/regions with different thicknesses to selectively control where deflection occurs. In the illustrated embodiment, for example, a first portion 713a of the membrane 713 has a first thickness, and a second portion 713b of the membrane 713 has a second thickness different than (e.g., less than) the first thickness. In other embodiments, however, the membrane 713 can include a different arrangement/configuration.

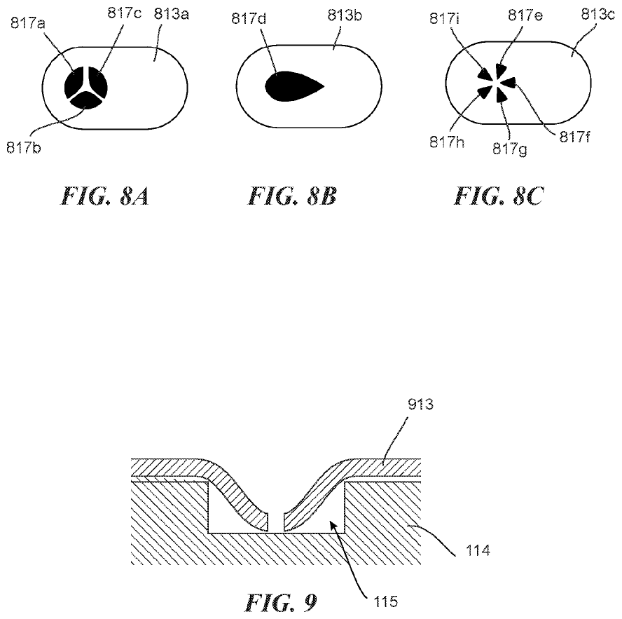

FIGS. 8A-8C schematically illustrate top views of portions of flexible membranes configured in accordance with further embodiments of the present technology. The disclosed flexible membranes can be similar to those described above with reference to FIGS. 1B and 3A-6E. The membranes of FIGS. 8A-8C, however, differ in that membranes have apertures with different shapes/arrangements. FIG. 8A, for example, schematically illustrates a membrane 813 having three non-circular apertures 817a-c arranged relative to each other. The apertures 817a-c are shaped and sized to allow fluid to flow therethrough, while providing additional structural support to the membrane 813 during operation. FIG. 8B illustrates a membrane 813b having an aperture 817d with a non-circular shape (e.g., a teardrop shape), and FIG. 8C illustrates a membrane 813c including a plurality of non-circular apertures 817e-i. It will be appreciated that membranes configured in accordance with further embodiments of the present technology can include one or more apertures having a variety of other suitable sizes, shapes, configurations, and/or arrangements.

FIG. 9 is a schematic, cross-sectional side view of a portion of a flexible membrane 913 configured in accordance with embodiments of the present technology. The membrane 913 is carried by the third layer 114 and positioned over well 115. The membrane 913 differs from those described previously in that membrane 913 is configured to deflect all the way to the floor of the well 115 (rather than only partially deflecting like the membranes described above with reference to FIGS. 3A-5C. One feature of the arrangement shown in FIG. 9 is that the floor of the well 115 can also be used to help with sealing during deflection of the membrane 913.

Without being bound by theory, the present technology is expected to improve performance of adjustable shunting systems, such as the system 100. In particular, the combination of the sealing assembly, the flexible membrane, and the well is expected to reduce or minimize leakage of fluid through the corresponding aperture when a user sets the actuator to the “closed” position. That is, the foregoing features are expected to improve the degree of control over fluid flow through adjustable shunting systems by providing a better “seal” when the actuator is moved to the “closed” position. In some embodiments, however, a small, fixed leak through the corresponding inlet may still exist even when the actuator has been moved to the “closed” position. For example, in some embodiments of the systems disclosed herein, the actuators can be tuned such that when they move to the “closed” position, they do not fully prevent fluid from flowing through the corresponding aperture, but instead permit reduced flow of fluid through the aperture (e.g., by substantially increasing the resistance through the aperture relative to when the actuator is in the “open” position).

The systems described herein can be designed for shunting fluid between a variety of body regions. As noted above, for example, in some embodiments the systems described herein are designed to be implanted in a patient's eye to shunt aqueous between the anterior chamber and a target outflow location (e.g., a subconjunctival bleb space), such as to treat glaucoma. Accordingly, in some embodiments the systems described herein can have dimensions compatible with being implanted in the patient's eye. For example, the systems described herein (e.g., the system 100) may have a length of between about 4 mm and about 20 mm, such as between about 4 mm and 15 mm, or between about 4 mm and 12 mm, or between about 6 mm and 10 mm, or about 8 mm. In some embodiments, the layers (e.g., the first layer 110, the second layer 112, and/or third layer 114) can have a width or thickness less than about 500 microns, less than about 400 microns, less than about 300 microns, and/or less than about 200 microns. In some embodiments, the diameter of the fluidic channels and corresponding apertures (e.g., the channels 104) may be less than about 100 microns, less than about 75 microns, and/or less than about 50 microns, such as about 35 microns. The foregoing dimensions are provided by way of example only, and other dimensions outside the ranges provided above are possible and included within the scope of the present technology. Indeed, the dimensions of the systems described herein may be designed depending on the type of shunting system (e.g., glaucoma shunt vs. hydrocephalus shunt) and intended recipient (e.g., child vs. adult).

### Examples

Several aspects of the present technology are set forth in the following examples:
1. An adjustable shunting system for shunting fluid from a first body region to a second body region within a patient, the system comprising:
a channel extending along an axial length of the adjustable shunting system, the channel providing a fluid flow path through the adjustable shunting system when the adjustable shunting system is implanted in the patient;
a well fluidly coupled to an end region of the channel, wherein the well has a first cross-sectional area;
a membrane extending over the well, wherein the membrane includes an aperture extending therethrough and configured to permit fluid to flow into the well, wherein the aperture has a second cross-sectional area that is less than the first cross-sectional area such that the membrane at least partially overlaps the well; and
an actuator having a sealing element configured to selectively control the flow of fluid through the aperture,
wherein—
   the membrane is positioned between the well and the actuator,
   the sealing element is moveable between (a) a first position in which the sealing element does not overlap with the well and does not cover the aperture, and (b) a second position in which the sealing element overlaps with the well and at least partially covers the aperture, and
   when the sealing element is in the second position, the membrane is configured to flex at least partially into the well.
2. The system of example 1 wherein, when the sealing element is in the second position, the sealing element is configured to seal the first aperture and prevent fluid from flowing therethrough.
3. The system of example 1 or example 2 wherein the sealing element is composed of a rigid material.
4. The system of any one of examples 1-3 wherein the sealing element has a spherical shape.
5. The system of any one of examples 1-4 wherein the actuator includes a sealing assembly, the sealing assembly comprising:
the sealing element, and
a sealing element retention feature,
wherein the sealing element is rotatably coupled to the sealing element retention feature.
6. The system of any one of examples 1-5 wherein the membrane has a thickness of between about 20 micron and about 80 micron.
7. The system of any one of examples 1-6 wherein the first cross-sectional area is between two and ten times greater than the second cross-sectional area.
8. The system of any one of examples 1-7 wherein:
the well has a first perimeter,
the aperture has a second perimeter, and
the first perimeter is between two to ten times greater than the second perimeter.
9. The system of any one of examples 1-8 wherein the aperture comprises a non-circular cross-sectional shape.
10. The system of any one of examples 1-9 wherein the aperture comprises a first aperture, and wherein the membrane comprises a second aperture extending therethrough.
11. The system of any one of examples 1-9 wherein the aperture comprises a first aperture, and wherein the membrane comprises a plurality of second apertures extending therethrough.
12. The system of any one of examples 1-11 wherein the well is circular, oval-shaped, and/or an elongated channel.
13. The system of any one of examples 1-12 wherein the membrane has a durometer of between about 70 and about 80.
14. The system of any one of examples 1-13 wherein, when the sealing element is in the second position, the membrane is configured to flex through the well such that a portion of the membrane engages a bottom surface of the well.
15. The system of any one of examples 1-14 wherein the well is a first well and the system further comprises a second well, and wherein:
the membrane extends over the second well; and
when the sealing element is in the first position, the sealing element overlaps the second well.
16. The system of example 15 wherein, when the sealing element is in the first position, the membrane is configured to flex at least partially into the second well.
17. The system of any one of examples 1-16, further comprising a surface extending over an opposite side of the actuator from the membrane, wherein the surface is stiffer than the membrane.
18. The system of example 17, further comprising a plate holding the actuator, wherein the plate includes the surface.
19. The system of example 17 wherein the surface includes a ramping feature configured to direct the sealing element into the well as the sealing element moves from the first position toward the second position.
20. The system of any one of examples 1-19 wherein the system is configured to shunt aqueous from an anterior chamber of the patient's eye to a target outflow location within the patient.
21. A method of controlling fluid flow through an implantable adjustable shunting system having an actuator carrying a sealing element, the method comprising:
actuating the actuator to move the sealing element toward an aperture in a membrane,
wherein the aperture has a first cross-sectional area and extends over a well having a second cross-sectional area greater than the first cross-sectional area such that the membrane at least partially covers the well, and
wherein, as the sealing element moves toward the aperture, the sealing element deflects the membrane at least partially into the well.
22. The method of example 21 wherein the aperture is centered above the well, and wherein the sealing element sits in and/or over the aperture to increase the fluid resistance therethrough after sealing element deflects the membrane at least partially into the well.
23. The method of example 21 or example 22 wherein actuating the actuator further causes the sealing element to engage a ramping feature, and wherein the ramping feature causes the sealing element to deflect the membrane at least partially into the well.

### Conclusion

The above detailed description of embodiments of the technology are not intended to be exhaustive or to limit the technology to the precise form disclosed above. Although specific embodiments of, and examples for, the technology are described above for illustrative purposes, various equivalent modifications are possible within the scope of the technology as those skilled in the relevant art will recognize. For example, any of the features of the intraocular shunts described herein may be combined with any of the features of the other intraocular shunts described herein and vice versa. Moreover, although steps are presented in a given order, alternative embodiments may perform steps in a different order. The various embodiments described herein may also be combined to provide further embodiments. For example, any of the various membrane configurations/arrangements described above with reference to FIGS. 6A-9 may be used with any of the other embodiments discussed previously with respect to FIGS. 1A-5C.

From the foregoing, it will be appreciated that specific embodiments of the technology have been described herein for purposes of illustration, but well-known structures and functions associated with intraocular shunts have not been shown or described in detail to avoid unnecessarily obscuring the description of the embodiments of the technology. Where the context permits, singular or plural terms may also include the plural or singular term, respectively.

Unless the context clearly requires otherwise, throughout the description and the examples, the words “comprise,” “comprising,” and the like are to be construed in an inclusive sense, as opposed to an exclusive or exhaustive sense; that is to say, in the sense of “including, but not limited to.” As used herein, the terms “connected,” “coupled,” or any variant thereof, means any connection or coupling, either direct or indirect, between two or more elements; the coupling of connection between the elements can be physical, logical, or a combination thereof. Additionally, the words “herein,” “above,” “below,” and words of similar import, when used in this application, shall refer to this application as a whole and not to any particular portions of this application. Where the context permits, words in the above Detailed Description using the singular or plural number may also include the plural or singular number respectively. As used herein, the phrase “and/or” as in “A and/or B” refers to A alone, B alone, and A and B. Additionally, the term “comprising” is used throughout to mean including at least the recited feature(s) such that any greater number of the same feature and/or additional types of other features are not precluded. It will also be appreciated that specific embodiments have been described herein for purposes of illustration, but that various modifications may be made without deviating from the technology. Further, while advantages associated with some embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

I/We claim:

1. An adjustable shunting system for shunting fluid from a first body region to a second body region within a patient, the system comprising:
a channel extending along an axial length of the adjustable shunting system, the channel providing a fluid flow path through the adjustable shunting system when the adjustable shunting system is implanted in the patient;
a well fluidly coupled to an end region of the channel, wherein the well has a first cross-sectional area;
a membrane extending over the well, wherein the membrane includes an aperture extending therethrough and configured to permit fluid to flow into the well, wherein the aperture has a second cross-sectional area that is less than the first cross-sectional area such that the membrane at least partially overlaps the well; and
an actuator having a sealing element configured to selectively control the flow of fluid through the aperture,
wherein—
the membrane is positioned between the well and the actuator,
the sealing element is moveable between (a) a first position in which the sealing element does not overlap with the well and does not cover the aperture, and (b) a second position in which the sealing element overlaps with the well and at least partially covers the aperture, and
when the sealing element is in the second position, the membrane is configured to flex at least partially into the well.

2. The system of claim 1 wherein, when the sealing element is in the second position, the sealing element is configured to seal the first aperture and prevent fluid from flowing therethrough.

3. The system of claim 1 wherein the sealing element is composed of a rigid material.

4. The system of claim 1 wherein the sealing element has a spherical shape.

5. The system of claim 1 wherein the actuator includes a sealing assembly, the sealing assembly comprising:
the sealing element, and
a sealing element retention feature,
wherein the sealing element is rotatably coupled to the sealing element retention feature.

6. The system of claim 1 wherein the membrane has a thickness of between about 20 micron and about 80 micron.

7. The system of claim 1 wherein the first cross-sectional area is between two and ten times greater than the second cross-sectional area.

8. The system of claim 1 wherein:
the well has a first perimeter,
the aperture has a second perimeter, and
the first perimeter is between two to ten times greater than the second perimeter.

9. The system of claim 1 wherein the aperture comprises a non-circular cross-sectional shape.

10. The system of claim 1 wherein the aperture comprises a first aperture, and wherein the membrane comprises a second aperture extending therethrough.

11. The system of claim 1 wherein the aperture comprises a first aperture, and wherein the membrane comprises a plurality of second apertures extending therethrough.

12. The system of claim 1 wherein the well is circular, oval-shaped, and/or an elongated channel.

13. The system of claim 1 wherein the membrane has a durometer of between about 70 and about 80.

14. The system of claim 1 wherein, when the sealing element is in the second position, the membrane is configured to flex through the well such that a portion of the membrane engages a bottom surface of the well.

15. The system of claim 1 wherein the well is a first well and the system further comprises a second well, and wherein:
the membrane extends over the second well; and
when the sealing element is in the first position, the sealing element overlaps the second well.

16. The system of claim 15 wherein, when the sealing element is in the first position, the membrane is configured to flex at least partially into the second well.

17. The system of claim 1, further comprising a surface extending over an opposite side of the actuator from the membrane, wherein the surface is stiffer than the membrane.

18. The system of claim 17, further comprising a plate holding the actuator, wherein the plate includes the surface.

19. The system of claim 17 wherein the surface includes a ramping feature configured to direct the sealing element into the well as the sealing element moves from the first position toward the second position.

20. The system of claim 1 wherein the system is configured to shunt aqueous from an anterior chamber of the patient's eye to a target outflow location within the patient.

21. A method of controlling fluid flow through an implantable adjustable shunting system having an actuator carrying a sealing element, the method comprising:
actuating the actuator to move the sealing element toward an aperture in a membrane,
wherein the aperture has a first cross-sectional area and extends over a well having a second cross-sectional area greater than the first cross-sectional area such that the membrane at least partially covers the well, and
wherein, as the sealing element moves toward the aperture, the sealing element deflects the membrane at least partially into the well.

22. The method of claim 21 wherein the aperture is centered above the well, and wherein the sealing element sits in and/or over the aperture to increase the fluid resistance therethrough after sealing element deflects the membrane at least partially into the well.

23. The method of claim 21 wherein actuating the actuator further causes the sealing element to engage a ramping feature, and wherein the ramping feature causes the sealing element to deflect the membrane at least partially into the well.
